(19) United States
(12) Patent Application Publication
(10) Pub. No.: US 2026/0217757 A1
(43) Pub. Date: Jul. 30, 2026

(54) SOLID PHASE PEPTIDE SYNTHESIS (SPPS) SOLVENT SYSTEM

(71) Applicant: Roche Diagnostics Operations, Inc., Indianapolis, IN (US)

(72) Inventors: Lars Hillringhaus, Königsdorf (DE); Nils Winter, München, Bavaria (DE)

(21) Appl. No.: 19/142,582

(22) PCT Filed: Dec. 21, 2023
(86) PCT No.: PCT/EP2023/087269

§ 371 Date: Jun. 23, 2025

(30) Foreign Application Priority Data
Dec. 23, 2022 (EP) 22216294.3

(51) Int. Cl. C07K 1/04

(52) U.S. Cl. CPC C07K 1/045

(57) ABSTRACT

The present invention relates to a solid phase peptide synthesis (SPPS) solvent system and use thereof. The present invention further relates to a solid phase peptide synthesis (SPPS), a kit, a peptide synthesizer and the use thereof.

### FIELD OF THE INVENTION

The present invention relates to a solid phase peptide synthesis (SPPS) solvent system and use thereof. The present invention further relates to a solid phase peptide synthesis (SPPS), a kit, a peptide synthesizer and the use thereof.

### BACKGROUND OF THE INVENTION

Peptides are of increasing interest in the pharmaceutical industry as potential drug candidates as well as components of diagnostic assays. Even though larger peptides can be expressed by recombinant methods, the majority of the marketed peptides are produced by chemical synthesis. The major technique herein is the so-called solid phase peptide synthesis (SPPS) that comprises the stepwise addition of protected amino acids to a growing peptide chain which is bound to a solid resin by a covalent bond. The advantage hereby is, that excess reagents and by-products can be removed from the reaction by a simple wash of the resin and therefore circumventing the need of intermediate purifications. The method involves repetitive cycles of coupling of the protected amino acid followed by removal of the protecting group and ultimately cleavage of the resin. Significant advances have been made within the aspects of protecting groups, coupling reagents, solid supports and the involvement of microwave-assisted systems to increase the temperature, therefore achieving higher reaction rates, better purity and shorter reaction times. Nevertheless, the later setup is of limited use for large-scale production and hampers the use of the more active uronium based activators.

As the synthesis of even medium sized peptides comprises a large number of synthetic steps there is a strong need for effective and high yielding reactions. This is usually assured by the use of a large excess of reagents but also requires the solvent to properly swell the resin used and to minimize aggregation of the growing peptide chain during assembly. Besides that, the solvent has to fully dissolve the e.g. Fmoc protected amino acid building blocks as well as the activators and all by-products generated during the reaction. The primary solvents that are commonly used in SPPS are N,N-dimethylformide (DMF), N,N-dimethylacetamide (DMA) and N-methylprrolidine (NMP). Alternative, environmentally friendlier solvents for the use in SPPS have been reported and include THE, MeCN (Org. Biomol. Chem., 2015, 13, 2393-2398, J. Pept. Sci. 2009, 15, 629-633), CPME (Amino Acids, 2016, 48, 419-426), 2-MeTHF (Amino Acids, 2016, 48, 419-426, ACS Sustainable Chem. Eng. 2016, 4, 6809-6814, Green Chem. Lett. Rev. 2021, 14, 153-164), NBP (Org. Process Res. Dev. 2018, 22, 494-503, Green Chem., 2020, 22, 3162-3169, Green Chem. Lett. Rev. 2021, 14, 153-164), γ-valerolactone (GVL) (Tetrahedron Lett. 2017, 58, 2986-2988, ACS Sustainable Chem. Eng. 2018, 6, 8034-8039.), propylene carbonate (PC) (Green Chem. 2017, 19, 1685-1691, Chem. Eur. J. 2019, 25, 4951-4964), 2,2,5,5-tetramethyloxolane (TMO) (Chem. Eur. J. 2019, 25, 4951-4964), dimethyl carbonat (DMC) (Green Chem. Lett. Rev. 2021, 14, 153-164), and N-formylmorpholine (Tetrahedron Lett. 2017, 58, 2986-2988) as well as mixtures of DMSO and toluene (J Pept. Res., 1997, 50, 2, 102-108), DMSO and 1,3-dioxolane (DOL) (Green Chem., 2021, 23, 3295-3311) DMSO and EtOAc (Green Chem., 2021, 23, 3295-3311, Green Chem., 2019, 21, 5990-5998) and DMSO and 2-MeTHF (Green Chem., 2021, 23, 3295-3311) as well as NBP and EtOAc (Green Chem., 2019, 21, 2594-2600, Green Chem., 2019, 21, 5990-5998.) and NBP and anisol (US 2019/0382438 A1).

Further attempts to render SPPS environmentally more friendly have been made by the development of alternative protecting groups, resins and activators that are compatible with the use of water as the reaction medium (Protein Pept. Lett. 2006, 13, 189-192, Org. Lett. 2012, 14, 3372-3375. Tetrahedron Lett., 2004, 45, 9293-9295, J. Org. Chem., 1978, 43, 4808-4816, Org. Lett., 2009, 11, 4488-4491, Green Chem., 2020, 22, 996). Nevertheless, drawbacks are associated with an outbalanced stability of the protecting group and lower yields and purities compared with conventional methods (ACS Sustainable Chem. Eng. 2019, 7, 3671-3683).

All previously described green alternatives for DMF, DMA and NMP in SPPS rely on the use of DIC at high temperatures or with prolonged reaction times and or give insufficient results using the more reactive uranium based activators.

Typically, amino acid building blocks and coupling reagents are used in a large excess to ensure completion of the reaction in a reasonable timeframe and significant amounts of solvent are necessary for effective washing of the resin. The solvent of choice is usually either N,N-dimethylformide (DMF), N,N-dimethylacetamide (DMA) or N-methylprrolidine (NMP) which are all classified as toxic for reproduction and therefore commercial peptide production generates large amounts of hazardous waste.

DMF, DMA and NMP are classified as environmentally problematic substances by the ECHA (European Chemicals Agency) under the REACH (Registration, Evaluation, Authorisation and Restriction of Chemicals) regulation in accordance with Article 57 (c). They were identified as SVHC (substances of very high concern) and are heading for restrictions (ECHA Annex XVII) and/or authorization for use (ECHA Annex XIV). This issue necessitates the replacements of these solvents in SPPS in the near future to avoid disruptions in the production of therapeutic and diagnostic peptides.

While all of the solvents proposed as green alternatives for DMF, DMA and NMP work somehow fine in combination with DIC, most of them suffer from poor solubility of uronium based activators like HATU. These are usually more reactive and therefore the first choice for synthesis performed at room temperature leading to significantly reduced coupling times. Furthermore, many solvents and mixtures thereof need high temperatures for a good performance which is often incompatible with the room temperature peptide-synthesisers used in many production sites.

Other drawbacks are the poor solubility of Fmoc amino acids in CPME, MeCN, EtOAc, DMC, PC and GVL as well as unwanted side reactions like ring-opening acylation of glycine residues with GVL and Arg-lactamisation in NBP in consequence of its high viscosity. The viscosity is not only a chemical problem but also leads to transfer issues in automated systems.

There is thus an urgent need in the art to overcome the above mentioned problems.

For the present invention, the solvent mixture is able to perform a standard DMF-protocol utilizing HATU in combination with short coupling times at room temperature.

It is an object of the present invention to provide a solid phase peptide synthesis (SPPS) solvent system and the use thereof. The present invention further relates to a solid phase peptide synthesis (SPPS), a kit, a peptide synthesizer and the use thereof.

This object is or these objects are solved by the subject matter of the independent claims. Further embodiments are subjected to the dependent claims.

### SUMMARY OF THE INVENTION

In the following, the present invention relates to the following apects:

In a first aspect, the present invention relates to a solid phase peptide synthesis (SPPS) solvent system comprising or consisting of a solvent mixture, wherein the solvent mixture comprises or consists of a first component, a second component and optionally a third component,
wherein the first component is acetonitrile (MeCN),
wherein the second component is dimethylsulfoxide (DMSO) or a tetrahydrofuran based solvent, and wherein the third component is the tetrahydrofuran based solvent in case of the second component is dimethylsulfoxide or wherein the third component is dimethylsulfoxide in case of the second component is the tetrahydrofuran based solvent.

In a second aspect, the present invention relates to the use of the solid phase peptide synthesis (SPPS) solvent system according to to the first aspect of the invention for solid phase peptide synthesis (SPPS).

In a third aspect, the present invention relates to a solid phase peptide synthesis (SPPS) comprises
A) a coupling step by coupling a carboxyl group of one amino acid unit to an amino group of another amino acid unit,

wherein the SPPS comprises the solid phase peptide synthesis (SPPS) solvent system according to the first aspect of the invention.

In a fourth aspect, the present invention relates to a kit comprises a solid phase peptide synthesis (SPPS) solvent system according to the first aspect of the invention.

In a fifth aspect, the present invention relates a the use of the kit according to the first aspect of the invention for a solid phase peptide synthesis (SPPS).

In a sixth aspect, the present invention relates to a peptide synthesizer comprises the solid phase peptide synthesis (SPPS) solvent system according to the first aspect of the invention.

In a seventh aspect, the present invention relates to the use of the peptide synthesizer of the sixth aspect of the invention for a solid phase peptide synthesis (SPPS).

### LIST OF FIGURES

FIG. 1 shows the general experimental procedure for solid phase peptide synthesis (SPPS).

FIG. 2 shows the SPPS solvent system according to the invention and in comparision exemplary embodiments.

FIGS. 3 to 36 show high performance liquid chromatograph of SPPS syntheses.

### DETAILED DESCRIPTION OF THE INVENTION

Before the present invention is described in detail below, it is to be understood that this invention is not limited to the particular embodiments and examples described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The various described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

Percentages, concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “4% to 20%” should be interpreted to include not only the explicitly recited values of 4% to 20%, but to also include individual values and sub-ranges within the indicated range. Thus, included in this numerical range are individual values such as 4, 5, 6, 7, 8, 9, 10, . . . 18, 19, 20% and sub-ranges such as from 4-10%, 5-15%, 10-20%, etc. This same principle applies to ranges reciting minimal or maximal values. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

In chemistry, “solid-phase synthesis” is a method in which molecules are covalently bound on a solid support material and synthesised step-by-step in a single reaction vessel utilising selective protecting group chemistry. As a specific embodiment, solid phase peptide synthesis is a common technique involving discrete steps for the synthesis of peptides. This approach permits unreacted reagents to be removed by washing without loss of product. Usually, peptides are synthesised from the carbonyl group side (C-terminus) to amino group side (N-terminus) of the amino acid chain. In peptide synthesis, an amino-protected amino acid is bound to a solid phase material such as, but not limited to, polystyrene beads, thereby forming a covalent bond between the carbonyl group and the resin, most often an amido or an ester bond. Then the amino group is deprotected and reacted with the carbonyl group of the next amino-protected amino acid. The solid phase now bears a dipeptide. This cycle is repeated to form the desired peptide chain. After all reactions are complete, the synthesised peptide is cleaved from the solid phase.

More specifically, the carboxyl moiety of each incoming amino acid is activated by one of several strategies and couples with the α-amino group of the preceding amino acid. The α-amino group of the incoming residue is temporarily blocked in order to prohibit peptide bond formation at this site. The residue is de-blocked at the beginning of the next synthesis cycle. In addition, reactive side chains on the amino acids are modified with appropriate protecting groups. The peptide chain is extended by reiteration of the synthesis cycle. Excess reagents are used to drive reactions as close to completion as possible.

“Solid phase peptide synthesis (SPPS)” is a well established method. Merrifield et al. were the first who developed a convenient strategy for the build up of peptides by subsequently coupling amino acid monomers using a solid phase resin as a heterogeneous reaction medium (R. B. Merrifield, J. Am. Chem. Soc. 85 (1963) 2149-2154).

As a major advantage in comparison with the in-solution synthesis of peptides SPPS can be automated easily and impurities or by-products, reagents as well as unreacted starting material can be washed away while the product or intermediate remains tethered on the solid phase.

Normally the abovementioned Merrifield method starts with the attachment of the first C-terminal amino acid to a so called “linker” of a crosslinked polystyrene resin. The “linker” serves as a bridging element between the resin and the C-terminal amino acid of the peptide to be synthesized and the linker contains an acid sensitive bond to be used for the detachment of the peptide after synthesis.

As an example for a typical SPPS protocol the N-terminus can be protected with the 9-fluorenylmethoxycarbonyl (Fmoc) group, which is stable in acid, but removable by base. Any side chain functional groups are protected with base stable groups to make sure that only the N-terminal amino group incorporated in the peptide backbone can react-after removal of the Fmoc group-with the carboxylic acid group of the subsequent amino acid. As already mentioned the first step after the immobilization of the first amino acid is the deprotection of the amino function by removal of the Fmoc group using 20% piperidine in N,N-dimethylformamide (DMF). The amino function is coupled with an activated carboxylic acid via 1-[Bis(dimethylamino)methylene]-1H-1,2,3-triazolo[4,5-b]pyridinium 3-oxide hexafluorophosphate (HATU) ester of the next amino acid in the presence of a base to form a new amide bond. This process is repeated until the desired peptide is assembled at the resin. As a last step the complete peptide is cleaved from the resin using a solution containing trifluoroacetic acid (TFA). The released peptide in the solution can be precipitated and washed before further purification.

This “classic” method for SPPS was optimized in recent years using modified resins, linkers, protective groups, coupling chemistries and cleavage procedures but the principle remains the same.

The term “solid phase” as used herein refers to a wide variety of materials including solids, semi-solids, membranes, particles, resins, papers and the like typically used by those of skill in the art to sequester molecules. The solid phase can be a material, e.g. a functionalized resin in a device for solid phase synthesis.

The “blocking group” or “protecting group” or “protection group” used for blocking the α-amino group determines both the synthesis chemistry employed and the nature of the side-chain protecting groups. The two most commonly used α-amino protecting groups are Fmoc (9-fluorenyl-methoxy-carbonyl) and Boc (tert-butoxycarbonyl). The protection of reactive groups in the side chains is provided by protecting groups which are orthogonal to the protecting group used for the α-amino group, which include but are not limited to carbamate, ether, ester, amide, acetal, enamine, thioether.

After fully assembling the peptide the side-chain protecting groups are removed, if so desired, and the peptide is cleaved from the solid support, using conditions that inflict minimal damage on labile residues.

The product can be analyzed to verify the sequence thereafter. A synthetic peptide is usually purified by HPLC or gel chromatography.

The term “peptide” means a molecule that is formed using naturally occurring L-amino acids or analogs thereof, like D-amino acids or N-alkylated amino acids or the like. Preferred amino acids are selected from the group consisting of Ala, Arg, Asn, Asp, Cys, Glu, Gln, Gly, His, Hyl, Hyp, Ile, Leu, Lys, Met, Phe, Pro, Ser, Thr, Trp, Tyr, and Val. Also other building blocks are possible having a carboxylic acid and an amino group. Additionally, modifications like fluorescence dyes or biotin are possible.

The term “room temperature” means a temperature of 20-25° C. In principle other temperatures are possible.

The term “washing step” can mean the treatment with sufficient amounts of reagent-free solvents to remove reagents and by-products.

The term “capping step” can mean acylation of unreacted amino-groups with a capping reagent before deprotection.

The term “deprotection step” can mean removal of the Fmoc-group by treatment with a base e.g. piperidine.

The term “cleavage step” can mean treatment of the peptide with an acid e.g. TFA and if necessary scavengersc, e.g. triisopropylsilane and water.

A “kit” is any manufacture (e.g., a package or container) comprising at least one reagent, e.g., a medicament for treatment of a disorder, or a probe for specifically detecting a biomarker gene or protein of the invention. The kit is preferably promoted, distributed, or sold as a unit for performing the methods of the present invention. Typically, a kit may further comprise carrier means being compartmentalized to receive in close confinement one or more container means such as vials, tubes, and the like. In particular, each of the container means comprises one of the separate elements to be used in the method of the first aspect. Kits may further comprise one or more other reagents including but not limited to reaction catalyst. Kits may further comprise one or more other containers comprising further materials including but not limited to buffers, diluents, filters, needles, syringes, and package inserts with instructions for use. A label may be present on the container to indicate that the composition is used for a specific application, and may also indicate directions for either in vivo or in vitro use. The computer program code may be provided on a data storage medium or device such as a optical storage medium (e.g., a Compact Disc) or directly on a computer or data processing device. Moreover, the kit may, comprise standard amounts for the biomarkers as described elsewhere herein for calibration purposes.

In this detailed description, references to “one embodiment”, “an embodiment”, or “in embodiments” mean that the feature being referred to is included in at least one embodiment of the technology with regards to all its aspects according to present disclosure. Moreover, separate references to “one embodiment”, “an embodiment”, or “embodiments” do not necessarily refer to the same embodiment; however, neither are such embodiments mutually exclusive, unless so stated, and except as will be readily apparent to those skilled in the art. Thus, the technology in all its aspects according to present disclosure can include any variety of combinations and/or integrations of the embodiments described herein.

### Embodiments

In a first aspect, the present invention relates to a solid phase peptide synthesis (SPPS) solvent system comprising or consisting of a solvent mixture, wherein the solvent mixture comprises or consists of a first component, a second component and optionally a third component,
wherein the first component is acetonitrile (MeCN),
wherein the second component is dimethylsulfoxide (DMSO) or a tetrahydrofuran based solvent, and wherein the third component is the tetrahydrofuran based solvent in case of the second component is dimethylsulfoxide or wherein the third component is dimethylsulfoxide in case of the second component is the tetrahydrofuran based solvent.

In particular, the SPPS solvent system is used in a solid phase peptide synthesis (SPPS), more preferably in the coupling step, washing step, capping step, deprotection step and/or cleavage step. More preferably, the SPPS solvent system is used in a solid phase peptide synthesis (SPPS) in the coupling step.

In embodiments of the first aspect of the invention, the SPPS solvent system or solvent mixture is able to perform a standard DMF-protocol utilizing HATU in combination with short coupling times at room temperature.

The coupling can be performed in a mixture of MeCN, 2-MeTHF and DMSO which is able to sufficiently dissolve all starting materials and by-products and ensures a high reaction rate in the coupling step. The deprotection and the washing step can be performed in DMSO as the single solvent. By using different solvent systems for these steps it can be ensure an optimal medium for each individual step circumventing the need of compromises. The advantage is that all solvents are in good agreement with current REACH related guidelines and therefore fulfilling the call of environmentally friendlier DMF substitutes.

In embodiments of the first aspect of the invention, the SPPS solvent system comprises the solvent mixture and other components, e.g. reagents for coupling, reagents for protection, reagents for deprotection, reagents for capping, reagents for labeling.

In embodiments of the first aspect of the invention, the SPPS solvent system consists of the solvent mixture.

In embodiments of the first aspect of the invention, the solvent mixture comprises a first component, a second component, preferably a third component and other components, e.g. reagents for coupling, reagents for protection, reagents for deprotection, reagents for capping, reagents for labeling. In embodiments of the first aspect of the invention, the solvent mixture consists of a first component and a second component.

In embodiments of the first aspect of the invention, the solvent mixture consists of a first component, a second component and a third component.

In embodiments of the first aspect of the invention, the tetrahydrofuran based solvent is Methyltetrahydrofuran (MeTHF). Methyltetrahydrofuran is less poisonal than e.g. THF.

In embodiments of the first aspect of the invention, the Methyltetrahydrofuran is 2-Methyltetrahydrofuran (2-MeTHF).

In embodiments of the first aspect of the invention, the Methyltetrahydrofuran is 3-Methyltetrahydrofuran (3-MeTHF).

In embodiments of the first aspect of the invention, the tetrahydrofuran based solvent is tetrahydrofuran (THF).

In embodiments of the first aspect of the invention, the tetrahydrofuran based solvent is substituted tetrahydrofuran.

In embodiments of the first aspect of the invention, said SPPS solvent system or solvent mixture is free of a solvent, which is selected from a group consisting of N,N-dimethylformamide (DMF), N,N-dimethylacetamide (DMA), N-methyl-2-pyrrolidon (NMP), dichloromethane (DCM) and mixtures thereof. Therefore, a less toxic and enviornmetla friendly solvent system can be provided.

In embodiments of the first aspect of the invention, said SPPS solvent system or solvent mixture is free of Diisopropylcarbodiimid (DIC). DIC does not show any efficient oligopeptide coupling at room temperature (RT).

In embodiments of the first aspect of the invention, the SPPS solvent mixture or solvent mixture comprises or consists of the first component and the second component,
wherein the first component is acetonitrile (MeCN), and
wherein the second component is dimethylsulfoxide (DMSO).

In embodiments of the first aspect of the invention, the SPPS solvent system or solvent mixture comprises or consists of the first component and the second component,
wherein the first component is acetonitrile (MeCN), and
wherein the second component is 2-methyltetrahydrofuran (2-MeTHF).

In embodiments of the first aspect of the invention, the SPPS solvent system or solvent mixture comprises or consists of the first component and the second component,
wherein the first component is acetonitrile (MeCN), and
wherein the second component is tetrahydrofuran (THF).

In embodiments of the first aspect of the invention, the SPPS solvent system or solvent mixture comprises or consists of the first component, the second component and the third component,
wherein the first component is acetonitrile (MeCN),
wherein the second component is dimethylsulfoxide (DMSO),
wherein the third component is a tetrahydrofuran based solvent, and
wherein the tetrahydrofuran based solvent is tetrahydrofuran (THF).

In embodiments of the first aspect of the invention, the SPPS solvent system or solvent mixture comprises or consists of the first component, the second component and the third component,
wherein the first component is acetonitrile (MeCN),
wherein the second component is dimethylsulfoxide (DMSO),
wherein the third component is a tetrahydrofuran based solvent, and
wherein the tetrahydrofuran based solvent is 2-methyltetrahydrofuran (2-MeTHF).

In embodiments of the first aspect of the invention, the SPPS solvent system or solvent mixture swells a resin, which is used for the solid phase peptide synthesis (SPPS).

In embodiments of the first aspect of the invention, the SPPS solvent system or solvent mixture reduces or minimize an aggregation of the peptide chain, in particular a growing peptide chain where a chain aggreagtes by itself or with neighboured chains.

In embodiments of the first aspect of the invention, the SPPS solvent system or solvent mixture dissolves the reagents and/or additives and/or by-products. Reagents are e.g. amino acid building blocks, coupling reagents, deptrotection reagents. Additives are e.g. formic acid and/or salts. By-products are e.g. ureas and/or dibenzofulvenes.

In embodiments of the first aspect of the invention, the SPPS solvent system or solvent mixture comprises or consists of the first component and the second component, wherein the ratio of the first component and the second component is in the range of from 4:1 to 2:3, preferably 3:2.

In embodiments of the first aspect of the invention, the SPPS solvent system or solvent mixture comprises a content of the first component in the range of 40 to 60 Vol %.

In embodiments of the first aspect of the invention, the SPPS solvent system or solvent mixture comprises or consists of the first component, the second component and the third component,
wherein the content of the first component is in the range from 40 Vol % to 80 Vol %,
wherein the content of the second component and optional third component are in the range from 20 Vol % to 60 Vol %,
wherein the content of the first, second and optional third components is 100 Vol %. The content of the first, second and optional third components is 100 Vol % can mean that the sum of the contents of the first, second and optional third components is 100 Vol %.

In embodiments of the first aspect of the invention, the content of the first component is selected form the group consisting of 40%, 45%, 50%, 55%, 60%, 65%, 70%, 75% and 80%.

In embodiments of the first aspect of the invention, the content of the second component is selected form the group consisting of 20 Vol %, 25 Vol %, 30 Vol %, 35 Vol %, 40 Vol %, 45 Vol %, 50 Vol %, 55 Vol % and 60 Vol %.

In embodiments of the first aspect of the invention, the content of the third component is selected form the group consisting of 20%, 25%, 30%, 35%, 40%, 45%, 50%, 55% and 60%.

In embodiments of the first aspect of the invention, the sum of the contents of the second and third components is 20% to 60%, preferably 40%.

In embodiments of the first aspect of the invention, the content of the second component is selected form the group consisting of 20 Vol %, 25%, 30%, 35%, 40%, 45%, 50%, 55% and 60%,
wherein the content of the third component is selected form the group consisting of 20%, 25%, 30%, 35%, 40%, 45%, 50%, 55% and 60%, and
wherein the sum of the contents of the second and third components is 20% to 60%.

In embodiments of the first aspect of the invention, the second component is DMSO, wherein the content of the second component is in the range of more than 0 to 60 Vol %, preferably 0.5 Vol % to 20 Vol %.

In embodiments of the first aspect of the invention, the second component is a tetrahydrofuran based solvent, wherein the content of the second component is in the range of 20 to 60 Vol %, preferably 22 Vol % to 55 Vol %.

In a second aspect, the present invention relates to the use of the solid phase peptide synthesis (SPPS) solvent system according to the first aspect of the invention for a solid phase peptide synthesis (SPPS).

All embodiments mentioned for the first aspect of the invention apply for the second aspect of the invention and vice versa.

In a third aspect, the present invention relates to a solid phase peptide synthesis (SPPS) comprises
A) a coupling step by coupling a carboxyl group of one amino acid unit to an amino group of another amino acid unit,
wherein the SPPS comprises the solid phase peptide synthesis (SPPS) solvent system according to the first aspect of the invention.

All embodiments mentioned for the first aspect of the invention and/or second aspect of the invention apply for the third aspect of the invention and vice versa.

In embodiments of the third aspect of the invention, the SPPS is used for diagnostic application.

In embodiments of the third aspect of the invention, the coupling step is performed at room temperature.

In embodiments of the third aspect of the invention, the coupling step is performed at a temperature that is selected from the range of 20° C. to 70° C., preferably 20° C. to 55° C., more preferably 20° C. to 35° C., more preferably 22° C. to 27° C., e.g. 25° C.

In embodiments of the third aspect of the invention, the coupling step comprises an activator, preferably a uronium based activator, more preferably [O-(7-Azabenzotriazol-1-yl)-N,N,N′,N′-tetramethyluronium-hexafluorphosphat] (HATU). In principle other activators are possible, e.g. phosphonium based or carbodiimid bases activators. These activators are known for a skilled person and are thus not explained in more detail. For example the activator can be ethyl cyano (hydroxyimino)acetate and/or DIC.

One advantage of the SPPS solvent system is that the coupling works with uronium-based coupling reagents such as HATU at room temperature.

In embodiments of the third aspect of the invention, the activator is not DIC.

In embodiments of the third aspect of the invention, the the activator is suitable to activate a carboxyl group, preferably by formation of an active ester.

In embodiments of the third aspect of the invention, the said SPPS comprises at least one of the following steps, which are performed after the coupling step:
B) Washing steps that are performed after steps A), C and/or D,
C) Optionally a capping step,
D) Deprotection step, and
E) Cleavage step.

In embodiments of the third aspect of the invention, the SPPS comprises more than one washing step. The washing steps are performed after each step, e.g. steps A), C and/or D.

In embodiments of the third aspect of the invention, the solid phase peptide synthesis (SPPS) is free of a solvent, which is selected from a group consisting of N,N-dimethylformamide (DMF), N,N dimethylacetamide (DMA), N-methyl-2-pyrrolidon (NMP), dichloromethane (DCM) and mixtures thereof.

In embodiments of the third aspect of the invention, the washing steps comprise or consist of DMSO as a single solvent.

In embodiments of the third aspect of the invention, the deprotection step comprises or consists of DMSO as a single solvent.

In embodiments of the third aspect of the invention, the cleavage step comprises or consists of TFA (Triflouro acidic acid), water and a further additive.

In embodiments of the third aspect of the invention, further additive are TIPS (triisopropylsilan) and/or EDT (ethandithiol).

In embodiments of the third aspect of the invention, the capping step comprises or consists of solvent components that are selected from the group consisting of 2-MeTHF, 3-MeTHF, THF, MeCN, DMSO and mixture of at least two of the solvent components.

In embodiments of the third aspect of the invention, the capping step comprises or consists of three solvent components, e.g. acetic anhydride, HATU and DIPEA.

In embodiments of the third aspect of the invention, the capping step comprises or consists of two solvent components, e.g. acetic anhydride and DIPEA.

In embodiments of the third aspect of the invention, the coupling step has a coupling time of 30 s to 180 min.

In embodiments of the third aspect of the invention, the solvent or solvent mixture in step A) is different from the solvent of steps B), D) and/or E).

In embodiments of the third aspect of the invention, the solvent or solvent mixture in step A) is the same solvent as used in step C).

In embodiments of the third aspect of the invention, the solvent or solvent mixture in step B) is the same solvent as used in step D), preferably DMSO as a single solvent.

In embodiments of the third aspect of the invention, step D) comprises a nitrogen containing base, e.g. piperidine.

In a fourth aspect, the present invention relates to a kit comprises a solid phase peptide synthesis (SPPS) solvent system according to the frist aspect of the invention.

All embodiments mentioned for the first aspect of the invention and/or second aspect of the invention and/or third aspect of the invention apply for the fourth aspect of the invention and vice versa.

In embodiments of the fourth aspect of the invention, the first component is stored in a first vessel,
wherein the second component is stored in a second vessel, and
optionally wherein the third component is stored in a third vessel.

In embodiments of the fourth aspect of the invention, the first component and the second component are stored in one vessel.

In embodiments of the fourth aspect of the invention, the first component and the second component and third component are stored in one vessel.

In embodiments of the fourth aspect of the invention, said kit comprises reagents like HATU, HOAt, DIPEA and/or additives like fomic acid, salts.

In embodiments of the fourth aspect of the invention, the kit is free of a solvent, which is selected from group consisting of N,N-dimethylformamide (DMF), N,N-dimethylacetamide (DMA), N-methyl-2-pyrrolidon (NMP), dichloromethane (DCM) and mixtures thereof.

In a fifth aspect, the present invention relates to the use of the kit according to the fourth aspect of the invention for a solid phase peptide synthesis (SPPS).

In embodiments of the fifth aspect of the invention, the kit is used for diagnostic application.

All embodiments mentioned for the first aspect of the invention and/or second aspect of the invention and/or third aspect of the invention and/or fourth aspect of the invention apply for the fifth aspect of the invention and vice versa.

In a sixth aspect, the present invention relates to a peptide synthesizer comprises the solid phase peptide synthesis (SPPS) solvent system according to the first aspect of the invention.

All embodiments mentioned for the first aspect of the invention and/or second aspect of the invention and/or third aspect of the invention and/or fourth aspect of the invention and/or fifth aspect of the invention apply for the sixth aspect of the invention and vice versa.

In a seventh aspect, the present invention relates to the use of the peptide synthesizer of the sixth aspect of the invention for a solid phase peptide synthesis (SPPS).

All embodiments mentioned for the first aspect of the invention and/or second aspect of the invention and/or third aspect of the invention and/or fourth aspect of the invention and/or fifth aspect of the invention and/or sixth aspect of the invention apply for the seventh aspect of the invention and vice versa.

In further embodiments, the present invention relates to the following aspects:
Embodiment 1. A solid phase peptide synthesis (SPPS) solvent system comprising or consisting of a solvent mixture, wherein the solvent mixture comprises or consists of a first component, a second component and optionally a third component,
wherein the first component is acetonitrile (MeCN),
wherein the second component is dimethylsulfoxide (DMSO) or a tetrahydrofuran based solvent, and wherein the third component is the tetrahydrofuran based solvent in case of the second component is dimethylsulfoxide or wherein the third component is dimethylsulfoxide in case of the second component is the tetrahydrofuran based solvent.
Embodiment 2. The SPPS solvent system of aspect 1, wherein the tetrahydrofuran based solvent is Methyltetrahydrofuran (MeTHF).
Embodiment 3. The SPPS solvent system of aspect 1 or 2, wherein the Methyltetrahydrofuran is 2-Methyltetrahydrofuran (2-MeTHF).
Embodiment 4. The SPPS solvent system of aspect 1 or 2, wherein the Methyltetrahydrofuran is 3-Methyltetrahydrofuran (3-MeTHF).
Embodiment 5. The SPPS solvent system of any of the preceding aspects, wherein the tetrahydrofuran based solvent is tetrahydrofuran (THF).
Embodiment 6. The SPPS solvent system of any of the preceding aspects, wherein the tetrahydrofuran based solvent is substituted tetrahydrofuran.
Embodiment 7. The SPPS solvent system of any of the preceding aspects, wherein said solvent system or solvent mixture is free of a solvent, which is selected from a group consisting of N,N-dimethylformamide (DMF), N,N-dimethylacetamide (DMA), N-methyl-2-pyrrolidon (NMP), dichloromethane (DCM) and mixtures thereof.
Embodiment 8. The SPPS solvent system of any of the preceding aspects, wherein said solvent system or solvent mixture is free of Diisopropylcarbodiimid (DIC).
Embodiment 9. The SPPS solvent system of any of the preceding aspects, wherein the solvent mixture comprises or consists of the first component and the second component,
wherein the first component is acetonitrile (MeCN), and
wherein the second component is dimethylsulfoxide (DMSO).
Embodiment 10. The SPPS solvent system of any of the preceding aspects, wherein the solvent mixture comprises or consists of the first component and the second component,
wherein the first component is acetonitrile (MeCN), and
wherein the second component is 2-methyltetrahydrofuran (2-MeTHF).
Embodiment 11. The SPPS solvent system of any of the preceding aspects, wherein the solvent mixture comprises or consists of the first component and the second component,
wherein the first component is acetonitrile (MeCN), and
wherein the second component is tetrahydrofuran (THF).
Embodiment 12. The SPPS solvent system of any of the preceding aspects, wherein the solvent mixture comprises or consists of the first component, the second component and the third component,
wherein the first component is acetonitrile (MeCN),
wherein the second component is dimethylsulfoxide (DMSO),
wherein the third component is a tetrahydrofuran based solvent, and
wherein the tetrahydrofuran based solvent is tetrahydrofuran (THF).
Embodiment 13. The SPPS solvent system of any of the preceding aspects, wherein the solvent mixture comprises or consists of the first component, the second component and the third component,
wherein the first component is acetonitrile (MeCN),
wherein the second component is dimethylsulfoxide (DMSO),
wherein the third component is a tetrahydrofuran based solvent, and
wherein the tetrahydrofuran based solvent is 2-methyltetrahydrofuran (2-MeTHF).
Embodiment 14. The SPPS solvent system of any of the preceding aspects, wherein the said solvent system or the solvent mixture swells a resin, which is used for the solid phase peptide synthesis (SPPS), e.g. for diagnostic application.
Embodiment 15. The SPPS solvent system of any of the preceding aspects, wherein the said solvent system or the solvent mixture reduces or minimize an aggregation of the peptide chain.
Embodiment 16. The SPPS solvent system of any of the preceding aspects, wherein the said solvent system or the solvent mixture dissolves the reagents and/or additives and/or by-products.
Embodiment 17. The SPPS solvent system of any of the preceding aspects, wherein the solvent mixture comprises or consists of the first component and the second component, wherein the ratio of the first component and the second component is in the range of from 4:1 to 2:3.
Embodiment 18. The SPPS solvent system of any of the preceding aspects, wherein the content of the first component is in the range of 40 to 60 Vol %.
Embodiment 19. The SPPS solvent system of any of the preceding aspects, wherein the solvent mixture comprises or consists of the first component, the second component and the third component,
wherein the content of the first component is in the range from 40 Vol % to 80 Vol %,
wherein the content of the second component and optional third component are in the range from 20 Vol % to 60 Vol %,
wherein the content of the first, second and optional third components is 100 Vol %. The content of the first, second and optional third components is 100 Vol % can mean that the sum of the contents of the first, second and optional third components is 100 Vol %.
Embodiment 20. The SPPS solvent system of any of the preceding aspects, wherein the content of the second component is selected form the group consisting of 20 Vol %, 25 Vol %, 30 Vol %, 35 Vol %, 40 Vol %, 45 Vol %, 50 Vol %, 55 Vol % and 60 Vol %.
Embodiment 21. The SPPS solvent system of any of the preceding aspects, wherein the content of the second component is selected form the group consisting of 20 Vol %, 25%, 30%, 35%, 40%, 45%, 50%, 55% and 60%,
wherein the content of the third component is selected form the group consisting of 20%, 25%, 30%, 35%, 40%, 45%, 50%, 55% and 60%, and
wherein the sum of the contents of the second and third components is 20% to 60%.
Embodiment 22. The SPPS solvent system of any of the preceding aspects, wherein the content of the first component is selected form the group consisting of 40%, 45%, 50%, 55%, 60%, 65%, 70%, 75% and 80%.
Embodiment 23. The SPPS solvent system of any of the preceding aspects, wherein second component is DMSO, wherein the content of the second component is in the range of more than 0 to 60 Vol %, preferably 0.5 Vol % to 20 Vol %.
Embodiment 24. The SPPS solvent system of any of the preceding aspects, wherein second component is a tetrahydrofuran based solvent, wherein the content of the second component is in the range of 20 to 60 Vol %, preferably 22 Vol % to 55 Vol %.
Embodiment 25. Use of the solid phase peptide synthesis (SPPS) solvent system according to at least one of the preceding aspects for solid phase peptide synthesis (SPPS).
Embodiment 26. A solid phase peptide synthesis (SPPS) comprises
   A) a coupling step by coupling a carboxyl group of one amino acid unit to an amino group of another amino acid unit,
   wherein the SPPS comprises the solid phase peptide synthesis (SPPS) solvent system according to at least one of the preceding aspects.
Embodiment 27. The SPPS of the preceding aspect, wherein the coupling step is performed at room temperature.
Embodiment 28. The SPPS of any of the preceding aspects, wherein the coupling step is performed at a temperature that is selected from the range of 20° C. to 70° C., preferably 20° C. to 55° C., more preferably 20° C. to 35° C., more preferably 22° C. to 27° C., e.g. 25° C.
Embodiment 29. The SPPS of any of the preceding aspects, wherein the coupling step comprises an activator, preferably a uronium based activator, more preferably [O-(7-Azabenzotriazol-1-yl)-N,N,N′,N′-tetramethyluronium-hexafluorphosphat] (HATU).
Embodiment 30. The SPPS of any of the preceding aspects, wherein the activator is suitable to activate a carboxyl group, preferably by formation of an active ester.
Embodiment 31. The SPPS of any of the preceding aspects, wherein said SPPS comprises at least one of the following steps, which are performed after the coupling step:
B) Washing steps that are performed after steps A), C and/or D,
C) Optionally a capping step,
D) Deprotection step, and
E) Cleavage step.
Embodiment 32. The SPPS of any of the preceding aspects, wherein the solid phase peptide synthesis (SPPS) is free of a solvent, which is selected from a group consisting of N,N-dimethylformamide (DMF), N,N dimethylacetamide (DMA), N-methyl-2-pyrrolidon (NMP), dichloromethane (DCM) and mixtures thereof.
Embodiment 33. The SPPS of any of the preceding aspects, wherein the SPPS of any of the preceding aspects, wherein the washing steps comprise or consist of DMSO as a single solvent.
Embodiment 34. The SPPS of any of the preceding aspects, wherein the deprotection step comprises or consists of DMSO as a single solvent.
Embodiment 35. The SPPS of any of the preceding aspects, wherein the cleavage step comprises or consists of TFA (Triflouro acidic acid), water and a further additive.
Embodiment 36. The SPPS of any of the preceding aspects, wherein the capping step comprises or consists of solvent components that are selected from the group consisting of 2-MeTHF, 3-MeTHF, THF, MeCN, DMSO and mixture of at least two of the solvent components.
Embodiment 37. The SPPS of any of the preceding aspects, wherein the coupling step has a coupling time of 30 s to 180 min.
Embodiment 38. The SPPS of any of the preceding aspects, wherein the solvent or solvent mixture in step A) is different from the solvent of steps B), D) and/or E).
Embodiment 39. The SPPS of any of the preceding aspects, wherein the solvent or solvent mixture in step A) is the same solvent as used in step C).
Embodiment 40. The SPPS of any of the preceding aspects, wherein the solvent or solvent mixture in step B) is the same solvent as used in step D), preferably DMSO as a single solvent.
Embodiment 41. The SPPS of any of the preceding aspects, wherein step D) comprises a nitrogen containing base, e.g. piperidine.
Embodiment 42. A kit comprises a solid phase peptide synthesis (SPPS) solvent system according to at least one of the preceding aspects.
Embodiment 43. The kit of the preceding aspect, wherein the first component is stored in a first vessel, wherein the second component is stored in a second vessel, and optionally wherein the third component is stored in a third vessel.
Embodiment 44. The kit of any of the preceding aspects, wherein the first component and the second component are stored in one vessel.
Embodiment 45. The kit of any of the preceding aspects, wherein the first component and the second component and third component are stored in one vessel.
Embodiment 46. The kit of any of the preceding aspects, wherein said kit comprises reagents and/or additives.
Embodiment 47. The kit of any of the preceding aspects, wherein the kit is free of a solvent, which is selected from group consisting of N,N-dimethylformamide (DMF), N,N-dimethylacetamide (DMA), N-methyl-2-pyrrolidon (NMP), dichloromethane (DCM) and mixtures thereof.
Embodiment 48. Use of the kit according to at least one of the preceding aspects for a solid phase peptide synthesis (SPPS).
Embodiment 49. A peptide synthesizer comprises the solid phase peptide synthesis (SPPS) solvent system according to at least one of the preceding aspects.
Embodiment 50. Use of the peptide synthesizer of the preceding aspect for a solid phase peptide synthesis (SPPS).

### Examples

The following examples are provided to illustrate, but not to limit the presently claimed invention.

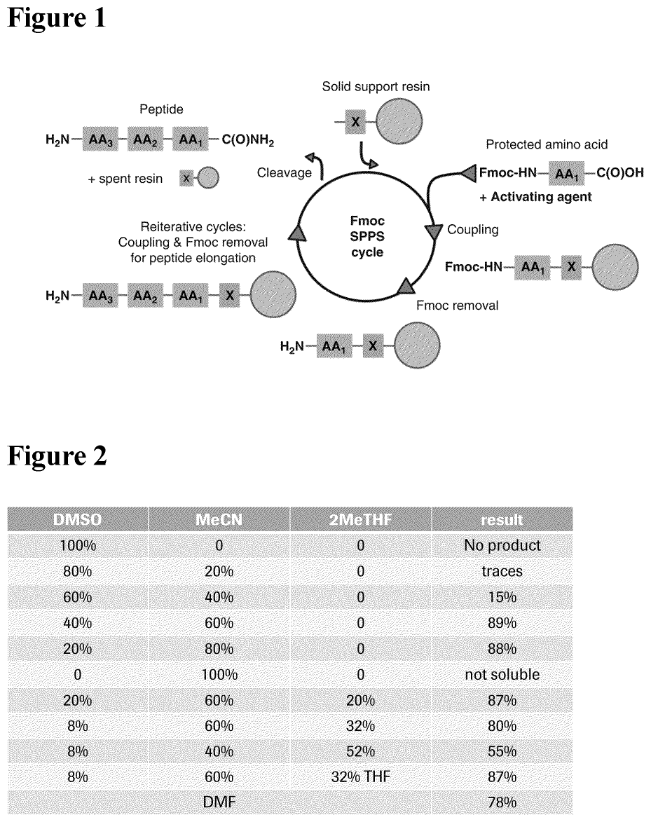

FIG. 1 shows the general experimental procedure for solid phase peptide synthesis (SPPS) with an example where Fmoc is removed. In principle other protecting groups can be used.

The synthesis was performed in an automatic peptide synthesizer (Prelude 6669, Gyros Protein Technologies) using the candidate resin (0.10 mmol scale). The resin was allowed to swell in 2-MeTHF (10 mL) for 20 min before the mixture was drained and repeatedly washed with the main solvent. The Fmoc protecting group was cleaved by threefold incubation with piperidine (20% in the main solvent) for 5 min each and the resin was subsequently washed repeatedly with the main solvent. The Fmoc protected amino acid building block (0.25M in SPPS solvent system 2 mL) was coupled using the SPPS solvent system (0.25M in SPPS solvent system, 2 mL) and DIPEA (1 M in the SPPS solvent system, 1 mL) for 5 min. After washing with the main solvent, a second coupling cycle was performed. The resin was repeatedly washed with the main solvent, capped for 5 min using the described capping system and washed again with the main solvent. The same cycle of washing, deprotection, coupling and capping was performed for every amino acid building block. After coupling and capping of the last amino acid, the resin was washed with the main solvent, the terminal Fmoc group was removed as described above and the resin was washed again with the main solvent.

The resin was washed with 2-MeTHF and transferred into a plastic syringe containing a PE frit. The peptide was cleaved from the resin by treating it with a mixture of TFA (9.5 mL), TIPS (0.25 mL) and water (0.25 mL) for 3 h at room temperature. The insoluble particles were removed by filtration and the residue was washed with TFA (2 mL). The liquid phase was concentrated to approximately half the volume by a stream of nitrogen, diisopropyl ether (80 mL) was added and the mixture was allowed to stand at 4° C. for 1 h. The precipitate was collected by filtration, washed with cold diisopropyl ether and dried on air. The remaining solids were dissolved in AcOH/water (1:5) and lyophilized to give the crude peptide as a colorless solid.

The peptides were analyzed on a C18 column using acetonitrile/water with 0.1% TFA as the solvent system.

FIG. 2 shows the SPPS for the peptide with the sequence IIKKSTALL using the solvent system according to the invention and in comparision exemplary embodiments. The results show that different mixing ratios are possible. Compared to known solvent systems, DMF is omitted without any disadvantages in purity.

Variation of the Solvent Composition in the Coupling Step (Sequence H₂N-IIKKSTALL-CONH₂; Peptide Comprises SEQ. No. 1 (IIKKSTALL))

### SPPS Solvent System for the Coupling Step:DMSO:MeCN: 2-MeTHF (2:8:0)

The peptide (sequence H₂N-IIKKSTALL-CONH₂) was synthesized by SPPS as described in the general procedure using a Tentagel R-RAM resin and DMSO as the main solvent. The amino acid building blocks were dissolved in 0.25M HOAt (part of HATU) in DMSO/MeCN=1:1, HATU (0.25M in MeCN) was used as the activator and DIPEA (1.0M in MeCN) was used as the base. Capping was performed by addition of AcOH (0.25M in 0.25M HOAt in DMSO/MeCN=1:1), HATU (0.25M in MeCN, 2 mL) and DIPEA (1.0M in MeCN, 1 mL) to the resin and incubation for 5 min at room temperature. The product was obtained as a colorless solid with a crude purity of 88%.

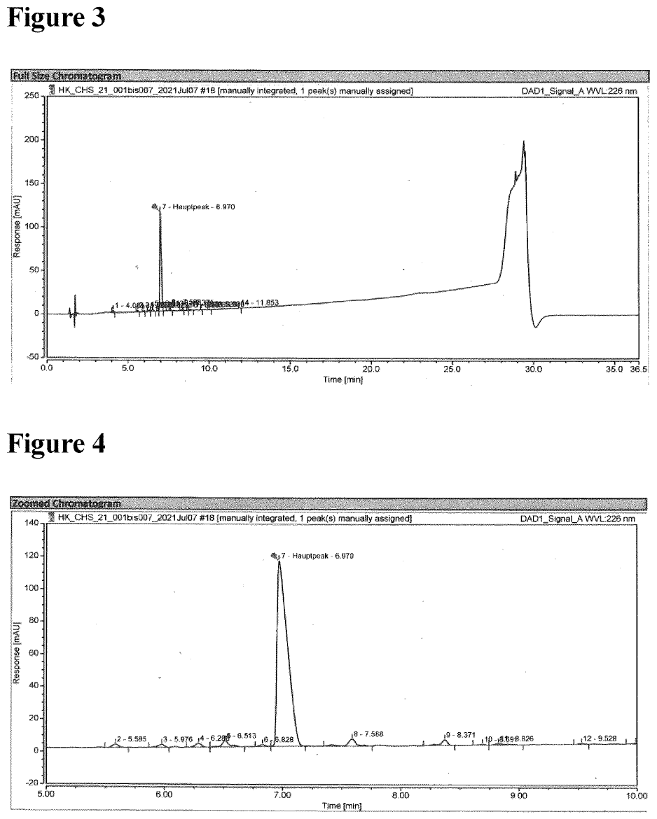

FIGS. 3 and 4 show high performance liquid chromatograph of SPPS synthesis of NH₂-IIKKSTALL-CONH₂ with DMSO:MeCN: 2-MeTHF (2:8:0) as the SPPS solvent system as described above. The purity of the product peak is 88%. The process using the current standard (DMF) gives a 78% pure product (reference). Therefore, the novel process performs better in terms of product purity circumventing the use mutagenic of teratogenic solvents.

### SPPS Solvent System for the Coupling Step: DMSO:MeCN:2-MeTHF (4:6:0)

The peptide (sequence H₂N-IIKKSTALL-CONH₂) was synthesized by SPPS as described in the general procedure using a Tentagel R-RAM resin and DMSO as the main solvent. The amino acid building blocks were dissolved in 0.25M HOAt in DMSO, HATU (0.25M in MeCN) was used as the activator and DIPEA (1.0M in MeCN) was used as the base. Capping was performed by addition of AcOH (0.25M in 0.25M HOAt in DMSO), HATU (0.25M in MeCN, 2 mL) and DIPEA (1.0M in MeCN, 1 mL) to the resin and incubation for 5 min at room temperature. The product was obtained as a colorless solid with a crude purity of 89%.

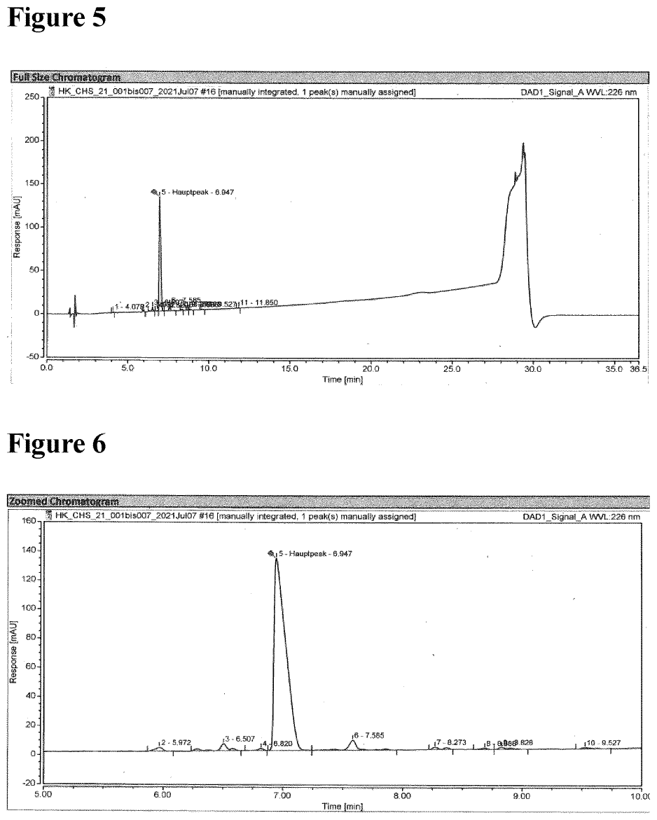

FIGS. 5 and 6 show high performance liquid chromatograph of SPPS synthesis of NH₂-IIKKSTALL-CONH₂ with DMSO:MeCN:2-MeTHF (4:6:0) as the coupling solvent or SPPS solvent system as described above. The purity of the product peak is 89%. The process using the current standard (DMF) gives a 78% pure product (reference). Therefore, the novel process performs better in terms of product purity circumventing the use mutagenic of teratogenic solvents.

### SPPS Solvent System for the Coupling Step: DMSO:MeCN:2-MeTHF (2:6:2)

The peptide (sequence H₂N-IIKKSTALL-CONH₂) was synthesized by SPPS as described in the general procedure using a Tentagel R-RAM resin and DMSO as the main solvent. The amino acid building blocks were dissolved in 0.25M HOAt in DMSO/2-MeTHF 1:1, HATU (0.25M in MeCN) was used as the activator and DIPEA (1.0M in MeCN) was used as the base. Capping was performed by addition of AcOH (0.25M in 0.25M HOAt in DMSO/2-MeTHF 1:1), HATU (0.25M in MeCN, 2 mL) and DIPEA (1.0M in MeCN, 1 mL) to the resin and incubation for 5 min at room temperature. The product was obtained as a colorless solid with a crude purity of 87%.

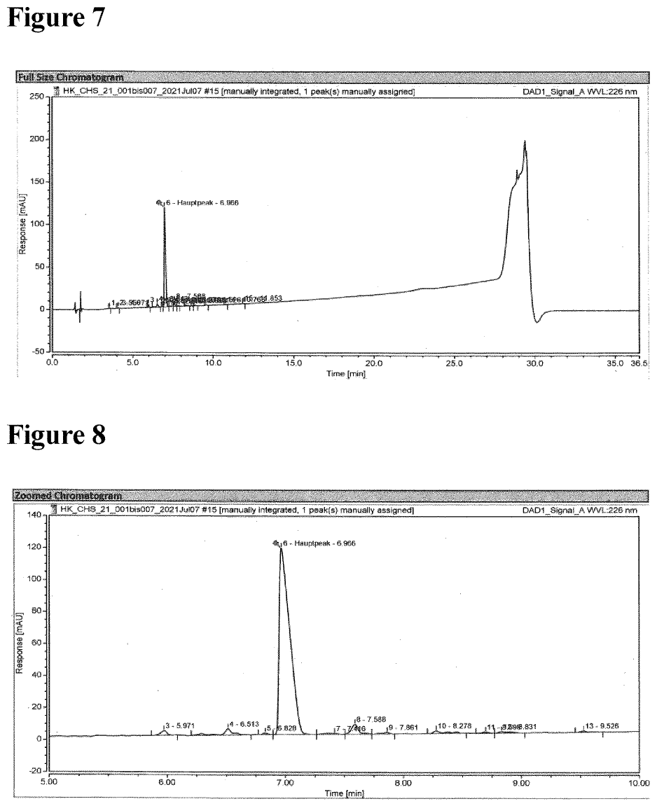

FIGS. 7 and 8 show high performance liquid chromatograph of SPPS synthesis of NH₂-IIKKSTALL-CONH₂ with DMSO:MeCN: 2-MeTHF (2:6:2) as the coupling solvent or SPPS solvent system as described above. The purity of the product peak is 87%. The process using the current standard (DMF) gives a 78% pure product (reference). Therefore, the novel process performs better in terms of product purity circumventing the use mutagenic of teratogenic solvents.

### SPPS Solvent System for the Coupling Step: DMSO:MeCN:2-MeTHF (8:60:32)

The peptide (sequence H₂N-IIKKSTALL-CONH₂) was synthesized by SPPS as described in the general procedure using a Tentagel R-RAM resin and DMSO as the main solvent. The amino acid building blocks were dissolved in 0.25M HOAt in DMSO/2-MeTHF 1:4, HATU (0.25M in MeCN) was used as the activator and DIPEA (1.0M in MeCN) was used as the base. Capping was performed by addition of AcOH (0.25M in 0.25M HOAt in DMSO/2-MeTHF 1:4), HATU (0.25M in MeCN, 2 mL) and DIPEA (1.0M in MeCN, 1 mL) to the resin and incubation for 5 min at room temperature. The product was obtained as a colorless solid with a crude purity of 70%.

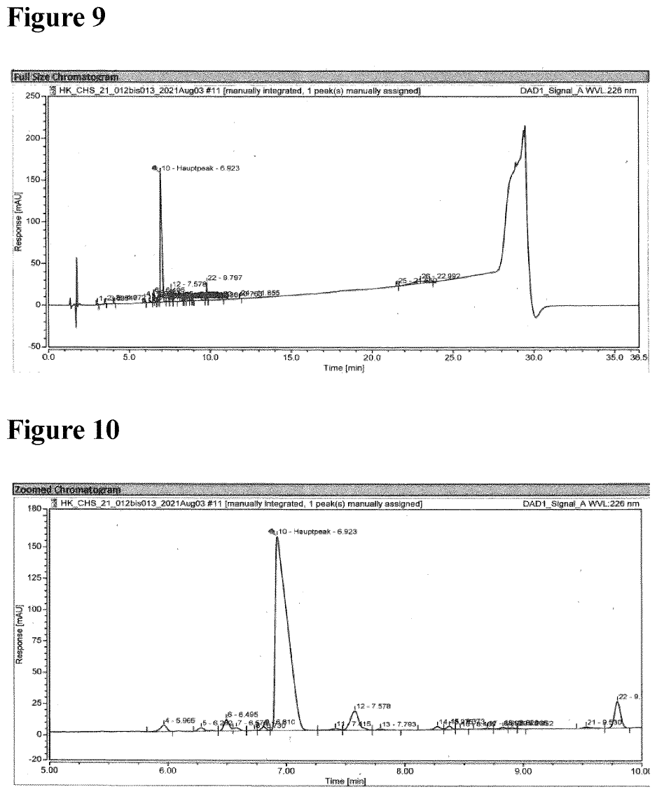

FIGS. 9 and 10 show high performance liquid chromatograph of SPPS synthesis of NH₂-IIKKSTALL-CONH₂ with DMSO:MeCN:2-MeTHF (8:60:32) as the coupling solvent or SPPS solvent system as described above. The purity of the product peak is 70%. The process using the current standard (DMF) gives a 78% pure product (reference). Therefore, the novel process performs in a comparably in terms of product purity circumventing the use of teratogenic solvents.

### SPPS Solvent System for the Coupling Step: DMSO:MeCN:THF (8:60:32))

The peptide (sequence H₂N-IIKKSTALL-CONH₂) was synthesized by SPPS as described in the general procedure using a Tentagel R-RAM resin and DMSO as the main solvent. The amino acid building blocks were dissolved in 0.25M HOAt in DMSO/THF 1:4, HATU (0.25M in MeCN) was used as the activator and DIPEA (1.0M in MeCN) was used as the base. Capping was performed by addition of AcOH (0.25M in 0.25M HOAt in DMSO/THF 1:4), HATU (0.25M in MeCN, 2 mL) and DIPEA (1.0M in MeCN, 1 mL) to the resin and incubation for 5 min at room temperature. The product was obtained as a colorless solid with a crude purity of 87%.

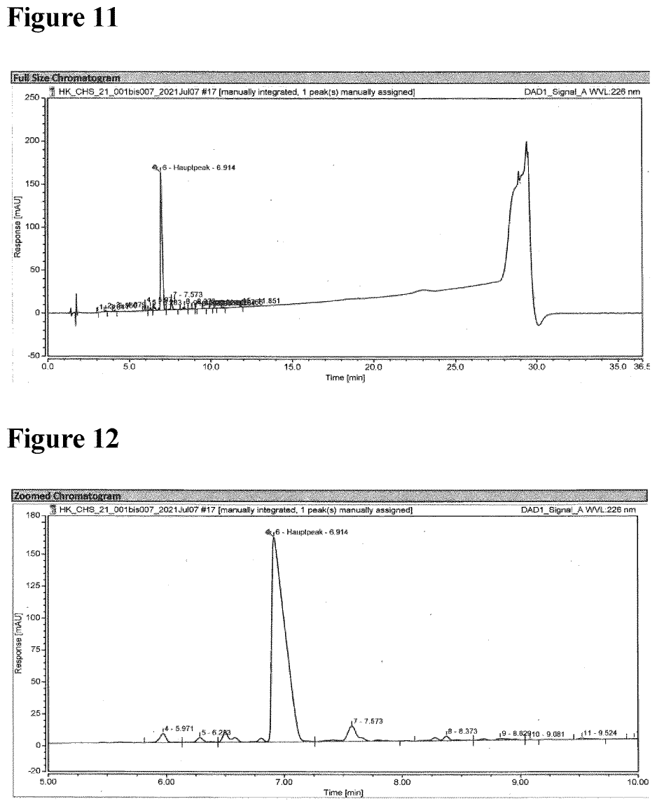

FIGS. 11 and 12 show high performance liquid chromatograph of SPPS synthesis of NH₂-IIKKSTALL-CONH₂ with DMSO:MeCN:THF (8:60:32) as the coupling solvent as described above. The purity of the product peak is 87%. The process using the current standard (DMF) gives a 78% pure product (reference). Therefore, the novel process performs better in terms of product purity circumventing the use of teratogenic solvents.

Variation of the Resin (Sequence H₂N-IIKKSTALL-CONH₂)

### Resin: ChemMatrix

The peptide (sequence H₂N-IIKKSTALL-CONH₂) was synthesized by SPPS as described in the general procedure using a ChemMatrix resin and DMSO as the main solvent. The amino acid building blocks were dissolved in 0.25M HOAt in DMSO/2-MeTHF 1:4, HATU (0.25M in MeCN) was used as the activator and DIPEA (1.0M in MeCN) was used as the base. Capping was performed by addition of AcOH (0.25M in 0.25M HOAt in DMSO/2-MeTHF 1:4), HATU (0.25M in MeCN, 2 mL) and DIPEA (1.0M in MeCN, 1 mL) to the resin and incubation for 5 min at room temperature. The product was obtained as a colorless solid with a crude purity of 63%. The SPPS solvent system is in this case DMSO, ACN and 2-MeTHF and results in the reactor when amino acids and reagents are mixed.

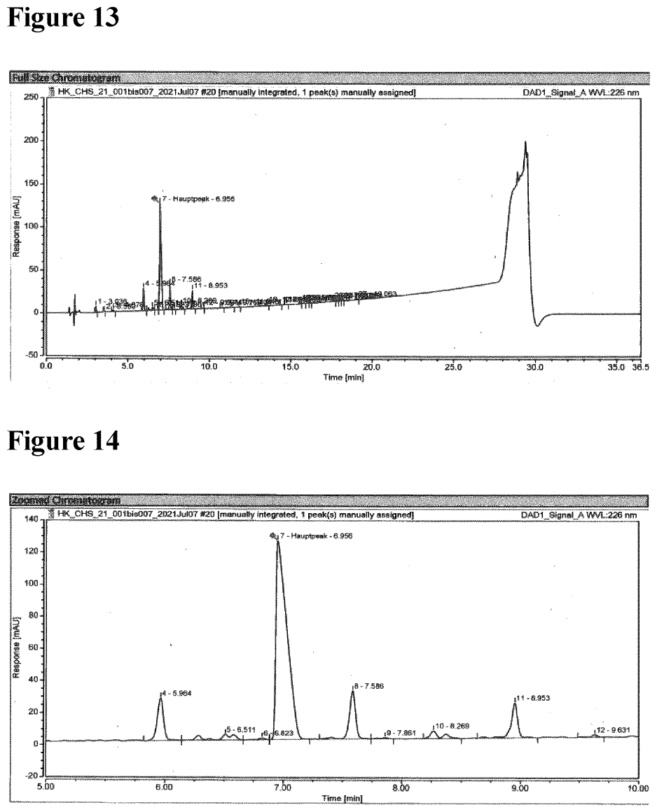

FIGS. 13 and 14 show high performance liquid chromatograph of SPPS synthesis of NH₂-IIKKSTALL-CONH₂ with DMSO:MeCN: 2-MeTHF (8:60:32) as the SPPS solvent system for the coupling step and a ChemMatrix resin as described above. The purity of the product peak is 63%. The experiment shows that the SPPS solvent system is not limited to one type of resin (Tentagel), but also works with other resins such as Chemmatrix.

### Resin: Rink-Amid SS/1% DVB

The peptide (sequence H₂N-IIKKSTALL-CONH₂) was synthesized by SPPS as described in the general procedure using a Rink-Amid SS/1% DVB resin and DMSO as the SPPS solvent system (1.ACN, 2./3. DMSO and Me-THF, respectively). The amino acid building blocks were dissolved in 0.25M HOAt in DMSO/2-MeTHF 1:4, HATU (0.25M in MeCN) was used as the activator and DIPEA (1.0M in MeCN) was used as the base. Capping was performed by addition of AcOH (0.25M in 0.25M HOAt in DMSO/2-MeTHF 1:4), HATU (0.25M in MeCN, 2 mL) and DIPEA (1.0M in MeCN, 1 mL) to the resin and incubation for 5 min at room temperature. The product was obtained as a colorless solid with a crude purity of 37%.

Peptide Sequence: H₂N-IIKKSTALL-CONH₂

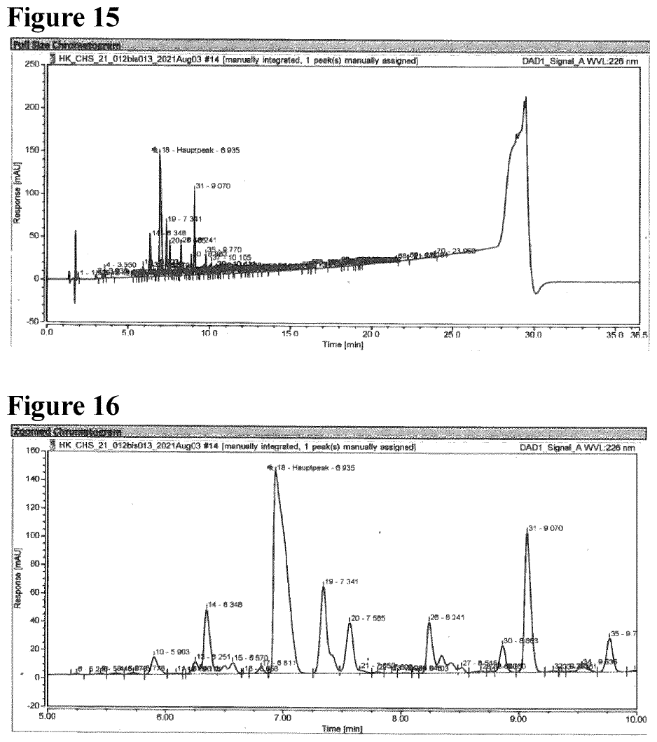

FIGS. 15 and 16 show high performance liquid chromatograph of SPPS synthesis of NH₂-IIKKSTALL-CONH₂ with DMSO:MeCN: 2-MeTHF (8:60:32) as the coupling solvent and a Rink-Amid SS/1% DVB resin as described above. The purity of the product peak is 37%. The experiment shows that the SPPS solvent system is not limited to one type of resin (Tentagel), but also works with other resins such as Chemmatrix (see above) or Rinkamid.

### Variation of the Activator and the Sequence

### Activator HBTU/HOBt

The peptide (sequence H₂N-IIKKSTALL-CONH₂) was synthesized by SPPS as described in the general procedure using a Tentagel R-RAM resin and DMSO as the SPPS solvent system. The amino acid building blocks were dissolved in 0.25M HOBt in DMSO/2-MeTHF 1:4, HBTU (0.25M in MeCN) was used as the activator and DIPEA (1.0M in MeCN) was used as the base. Capping was performed by addition of AcOH (0.25M in 0.25M HOBt in DMSO/2-MeTHF 1:4), HBTU (0.25M in MeCN, 2 mL) and DIPEA (1.0M in MeCN, 1 mL) to the resin and incubation for 5 min at room temperature. The product was obtained as a colorless solid with a crude purity of 92%.

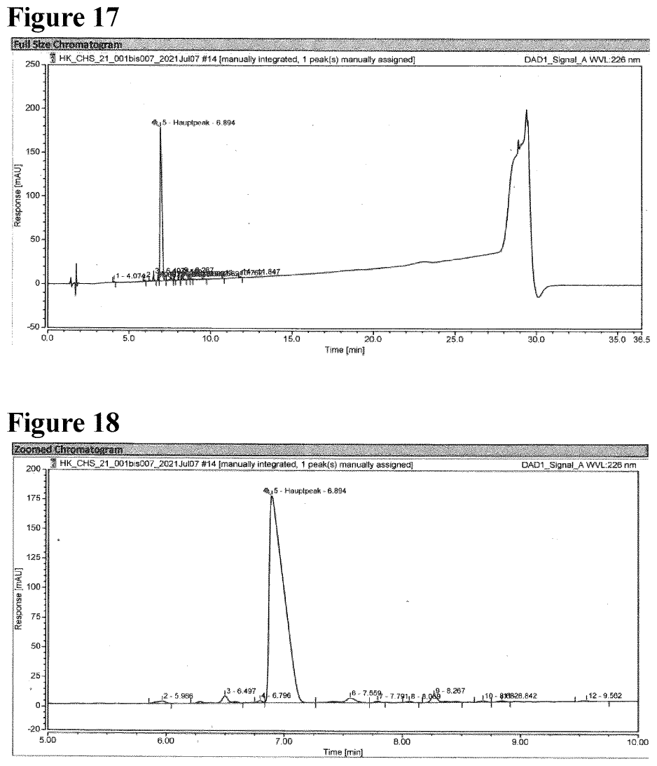

FIGS. 17 and 18 show high performance liquid chromatograph of SPPS synthesis of NH₂-IIKKSTALL-CONH₂ with DMSO:MeCN: 2-MeTHF (8:60:32) as the coupling solvent and HBTU/HOBt as the activator system as described above. The purity of the product peak is 92%. The experiment shows that the solvent system is not limited to one coupling reagent (HATU/HOAt), but also works with other coupling reagents such as HBTU/HOBt.

Activator DIC/Oxyma Pure, Sequence: NH₂-VHLTK(Cbz)-CONH₂

The peptide (sequence NH₂-VHLTK(Cbz)-CONH₂; peptide comprises SEQ. No. 2 (VHLTK) and Cbz, wherein Cbz is benzyloxycarbonyl) was synthesized by SPPS as described in the general procedure using a Tentagel R-RAM resin and DMSO as the SPPS solvent system. The amino acid building blocks were dissolved in 0.25M Oxyma Pure (ethyl cyano (hydroxyimino)acetate) in DMSO/2-MeTHF 1:4, DIC (0.25M in MeCN) was used as the activator and DIPEA (1.0M in MeCN) was used as the base. Capping was performed by addition of Ac₂O (0.13M in 0.50M DIPEA in DMSO/2-MeTHF 1:4) to the resin and incubation for 5 min at room temperature. The product was obtained as a colorless solid with a crude purity of 50%.

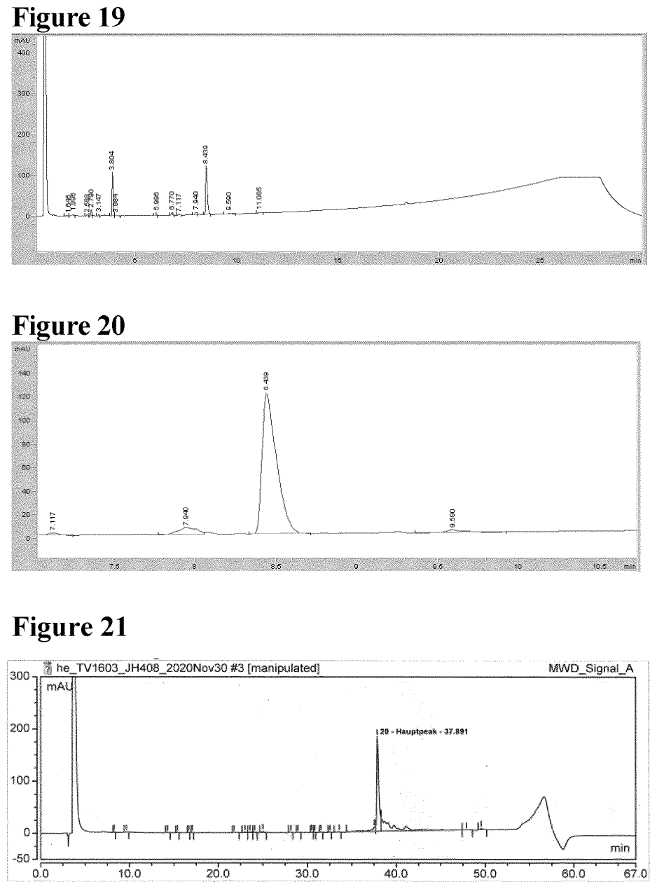

FIGS. 19 and 20 show high performance liquid chromatograph of SPPS synthesis of NH₂-VHLTK(Cbz)-CONH₂ with DMSO:MeCN: 2-MeTHF (8:60:32) as the coupling solvent and DIC/Oxyma pure as the activator system as described above. The purity of the product peak is 50%. The experiment shows that the solvent system is not limited to one coupling reagent (HATU/HOAt), but also works with other coupling reagents such as DIC/Oxyma.

Activator HATU/HOAt, sequence: NH₂-CbAlaAcpbAlaKKNKRNTNRRPQDVKFPGGGQIVGGVYLLPR RGPRLGVRA-CONH₂ (peptide comprises SEQ. No. 3 (KKNKRNTNRRPQDVKFPGGGQIVGGVYLLPR RGPRLGVRA) and CbAlaAcpbAla, wherein CbAlaAcpbAla means Cysteine-β-Alanin-6-Aminocaproic acid-β-Alanin.

The peptide (sequence NH₂-CbAlaAcpbAlaKKNKRNTNRRPQDVKFPGGGQIVGGVYLLPR RGPRLGVRA-CONH₂) was synthesized by SPPS as described in the general procedure using a Tentagel R-RAM resin and DMSO as the SPPS solvent system. The amino acid building blocks were dissolved in 0.25M HOAt in DMSO/2-MeTHF 1:4, HATU (0.25M in MeCN) was used as the activator and DIPEA (1.0M in MeCN) was used as the base. Capping was performed by addition of AcOH (0.25M in 0.25M HOAt in DMSO/2-MeTHF 1:4), HATU (0.25M in MeCN, 2 mL) and DIPEA (1.0M in MeCN, 1 mL) to the resin and incubation for 5 min at room temperature. The product was obtained as a colorless solid with a crude purity of 49%.

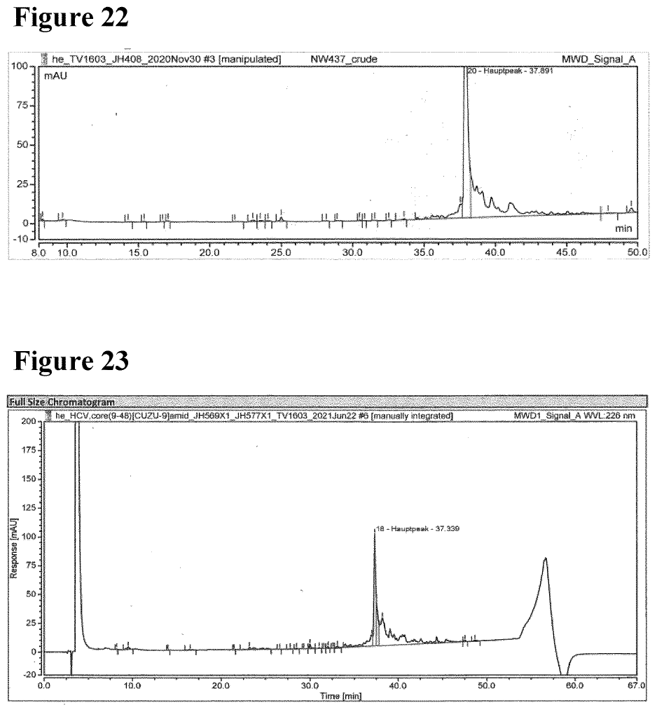

FIGS. 21 and 22 show high performance liquid chromatograph of SPPS synthesis of NH₂-CbAlaAcpbAlaKKNKRNTNRRPQDVKFPGGGQIVGGVYLLPR RGPRLGVRA-CONH₂ with DMSO:MeCN: 2-MeTHF (8:60:32) as the coupling solvent and HATU/HOAt as the activator system as described above. The purity of the product peak is 49%. The experiment shows the application of the solvent system for a different peptide sequence (HCV) instead of IIKKSTALL.

Activator HATU, sequence: NH₂-CbAlaAcpbAlaKKNKRNTNRRPQDVKFPGGGQIVGGVYLLPR RGPRLGVRA-CONH₂

The peptide (sequence NH₂-CbAlaAcpbAlaKKNKRNTNRRPQDVKFPGGGQIVGGVYLLPR RGPRLGVRA-CONH₂) was synthesized by SPPS as described in the general procedure using a Tentagel R-RAM resin and DMSO as the SPPS solvent system. The amino acid building blocks were dissolved in DMSO/2-MeTHF 1:4, HATU (0.25M in MeCN) was used as the activator and DIPEA (1.0M in MeCN) was used as the base. Capping was performed by addition of AcOH (0.25M in DMSO/2-MeTHF 1:4), HATU (0.25M in MeCN, 2 mL) and DIPEA (1.0M in MeCN, 1 mL) to the resin and incubation for 5 min at room temperature. The product was obtained as a colorless solid with a crude purity of 25%.

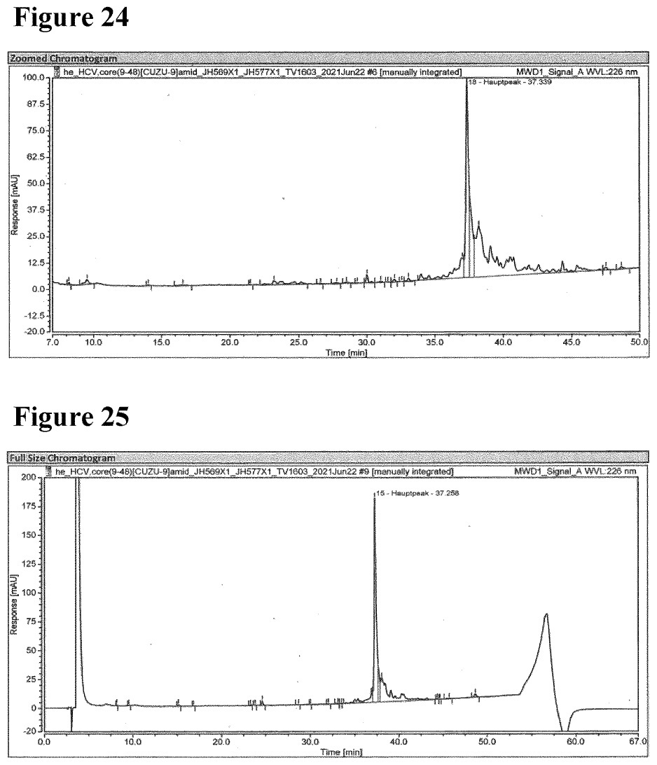

FIGS. 23 and 24 show High performance liquid chromatograph of SPPS synthesis of NH₂-CbAlaAcpbAlaKKNKRNTNRRPQDVKFPGGGQIVGGVYLLPR RGPRLGVRA-CONH₂ with DMSO:MeCN:THF (8:60:32) as the coupling solvent and HATU as the activator system as described above. The purity of the product peak is 25%. It is shown the synthesis uses only HATU as coupling reagent instead of HATU/HOAt.

Activator HATU/Oxyma Pure, sequence: NH₂-CbAlaAcpbAlaKKNKRNTNRRPQDVKFPGGGQIVGGVYLLPR RGPRLGVRA-CONH₂

The peptide (sequence NH₂-CbAlaAcpbAlaKKNKRNTNRRPQDVKFPGGGQIVGGVYLLPR RGPRLGVRA-CONH₂) was synthesized by SPPS as described in the general procedure using a Tentagel R-RAM resin and DMSO as the SPPS solvent system. The amino acid building blocks were dissolved in 0.25M Oxyma Pure in DMSO/2-MeTHF 1:4, HATU (0.25M in MeCN) was used as the activator and DIPEA (1.0M in MeCN) was used as the base. Capping was performed by addition of AcOH (0.25M in 0.25M Oxyma Pure in DMSO/2-MeTHF 1:4), HATU (0.25M in MeCN, 2 mL) and DIPEA (1.0M in MeCN, 1 mL) to the resin and incubation for 5 min at room temperature. The product was obtained as a colorless solid with a crude purity of 54%.

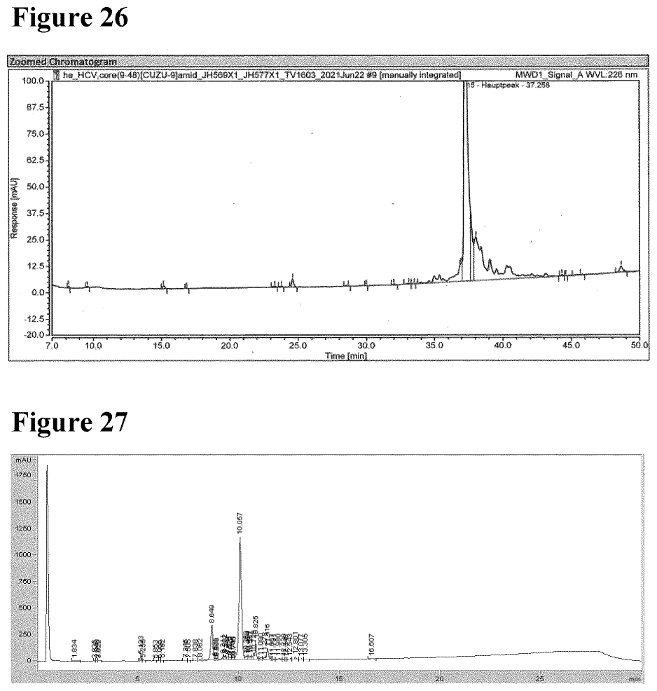

FIGS. 25 and 26 show high performance liquid chromatograph of SPPS synthesis of NH₂-CbAlaAcpbAlaKKNKRNTNRRPQDVKFPGGGQIVGGVYLLPR RGPRLGVRA-CONH₂ with DMSO:MeCN: 2-MeTHF (8:60:32) as the coupling solvent and HATU/Oxyma as the activator system as described above. The purity of the product peak is 54%. It is shown the synthesis with HATU/Oxyma as coupling reagent instead of HATU/HOAt.

Activator:HATU/HOAt, sequence: NH₂-VYWTSPFMKLIHEQCNRADG-CONH₂

The peptide (NH₂-VYWTSPFMKLIHEQCNRADG-CONH₂; peptide comprises SEQ. No. 4 (VYWTSPFMKLIHEQCNRADG)) was synthesized by SPPS as described in the general procedure using a Tentagel R-RAM resin and DMSO as the SPPS solvent system. The amino acid building blocks were dissolved in 0.25M HOAt in DMSO/2-MeTHF 1:4, HATU (0.25M in MeCN) was used as the activator and DIPEA (1.0M in MeCN) was used as the base. Capping was performed by addition of AcOH (0.25M in 0.25M HOAt in DMSO/2-MeTHF 1:4), HATU (0.25M in MeCN, 2 mL) and DIPEA (1.0M in MeCN, 1 mL) to the resin and incubation for 5 min at room temperature. The product was obtained as a colorless solid with a crude purity of 55%.

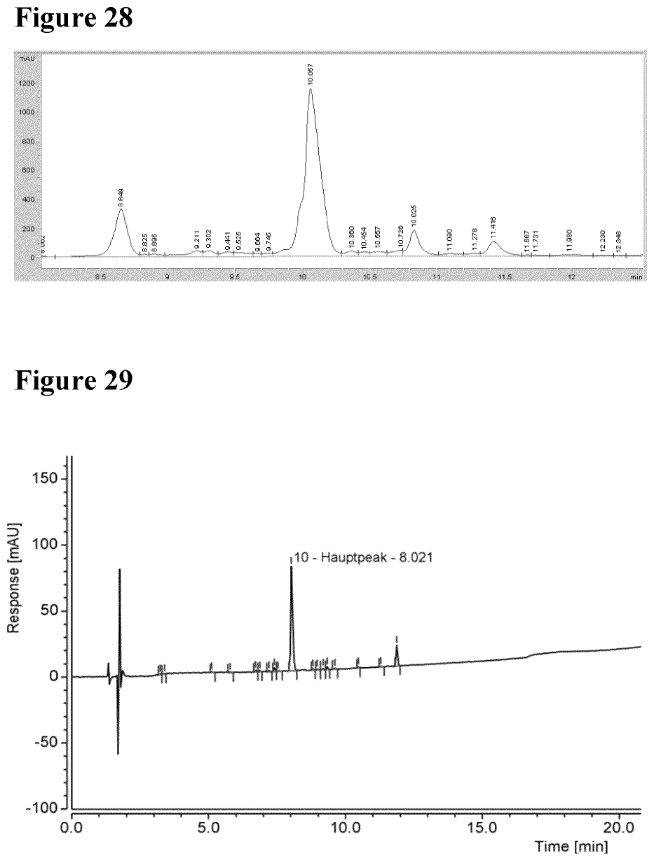

FIGS. 27 and 28 show high performance liquid chromatograph of SPPS synthesis of NH₂-VYWTSPFMKLIHEQCNRADG-CONH₂ with DMSO:MeCN: 2-MeTHF (8:60:32) as a solvent and a Tentagel R-RAM resin as described above. The purity of the product peak is 87%. The process using the current standard (DMF) gives a 55% pure product (reference). Therefore, the novel process performs better in terms of product purity circumventing the use of teratogenic solvents.

Variation of the Temperature in the Coupling Step (Sequence H₂N-IIKKSTALL-CONH₂; Peptide Comprises SEQ. No. 1 (IIKKSTALL); Activator DIC/Oxyma Pure; 50° C.)

The peptide (sequence H₂N-IIKKSTALL-CONH₂) was synthesized by SPPS using a ProTide LL resin and DMSO as SPPS main solvent. The synthesis was performed on an automatic peptide synthesizer (Liberty Blue, CEM Corporation). The resin was allowed to swell in DMSO (6 mL for 10 min) before the mixture was drained and repeatedly washed with the main solvent. The Fmoc protecting group was cleaved by incubation with piperidine (20% in the main solvent with 5% formic acid) for 3 min and the resin was subsequently washed repeatedly with the main solvent. The Fmoc-protected amino acid building blocks were dissolved in in DMSO/2-MeTHF 1:4. DIC (0.25M in MeCN) and Oxyma Pure (0.25M in MeCN) were used as the activators. Coupling was carried out for 16.7 min at 50° C. The resin was repeatedly washed with the main solvent. Capping was performed by addition of Ac₂O (0.13M in DMSO/2-MeTHF 1:4) to the resin and incubation for 2×1 min and washed again with the main solvent. The same cycle of washing, deprotection, coupling and capping was performed for every amino acid building block. After coupling and capping of the last amino acid, the resin was washed with the main solvent, the terminal Fmoc group was removed as described above and the resin was washed again with the main solvent. The resin was washed with 2-MeTHF and transferred into a plastic syringe containing a PE frit. The peptide was cleaved from the resin by treating it with a mixture of TFA (18 mL), TIPS (1 mL) and water (1 mL) for 2.5 h at room temperature. Cold diisopropyl ether (150 mL) was added. The precipitate was collected by filtration, washed with cold diisopropyl ether and dried on air. The remaining solids were dissolved in AcOH/water (1:5) and lyophilized to give the crude peptide as a colorless solid. The peptides were analyzed on a C18 column using acetonitrile/water with 0.1% TFA as the solvent system. The product was obtained as a colorless solid with a crude purity of 76%.

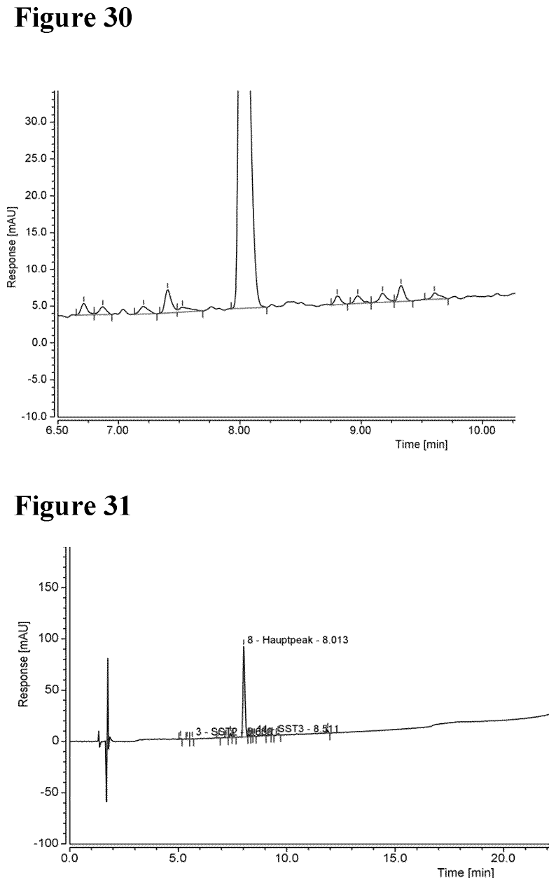

FIGS. 29 and 30 show high performance liquid chromatograph of SPPS synthesis of H₂N-IIKKSTALL-CONH₂ with DMSO:MeCN: 2-MeTHF (8:60:32) as the coupling solvent and DIC/Oxyma pure as the activator system at 50° C. as described above. The purity of the product peak is 76%. The experiment shows that the solvent system is not limited to room temperature but also works at 50° C.

Variation of the Temperature in the Coupling Step (Sequence H₂N-IIKKSTALL-CONH₂; Peptide Comprises SEQ. No. 1 (IIKKSTALL); Activator DIC/Oxyma Pure; 60° C.)

The peptide (sequence H₂N-IIKKSTALL-CONH₂) was synthesized by SPPS using a ProTide LL resin and DMSO as SPPS main solvent. The synthesis was performed on an automatic peptide synthesizer (Liberty Blue, CEM Corporation). The resin was allowed to swell in DMSO (6 mL for 10 min) before the mixture was drained and repeatedly washed with the main solvent. The Fmoc protecting group was cleaved by incubation with piperidine (20% in the main solvent with 5% formic acid) for 3 min and the resin was subsequently washed repeatedly with the main solvent. The Fmoc-protected amino acid building blocks were dissolved in in DMSO/2-MeTHF 1:4. DIC (0.25M in MeCN) and Oxyma Pure (0.25M in MeCN) were used as the activators. Coupling was carried out for 16.7 min at 60° C. The resin was repeatedly washed with the main solvent. Capping was performed by addition of Ac₂O (0.13M in DMSO/2-MeTHF 1:4) to the resin and incubation for 2×1 min and washed again with the main solvent. The same cycle of washing, deprotection, coupling and capping was performed for every amino acid building block. After coupling and capping of the last amino acid, the resin was washed with the main solvent, the terminal Fmoc group was removed as described above and the resin was washed again with the main solvent. The resin was washed with 2-MeTHF and transferred into a plastic syringe containing a PE frit. The peptide was cleaved from the resin by treating it with a mixture of TFA (18 mL), TIPS (1 mL) and water (1 mL) for 2.5 h at room temperature. Cold diisopropyl ether (150 mL) was added. The precipitate was collected by filtration, washed with cold diisopropyl ether and dried on air. The remaining solids were dissolved in AcOH/water (1:5) and lyophilized to give the crude peptide as a colorless solid. The peptides were analyzed on a C18 column using acetonitrile/water with 0.1% TFA as the solvent system. The product was obtained as a colorless solid with a crude purity of 86%.

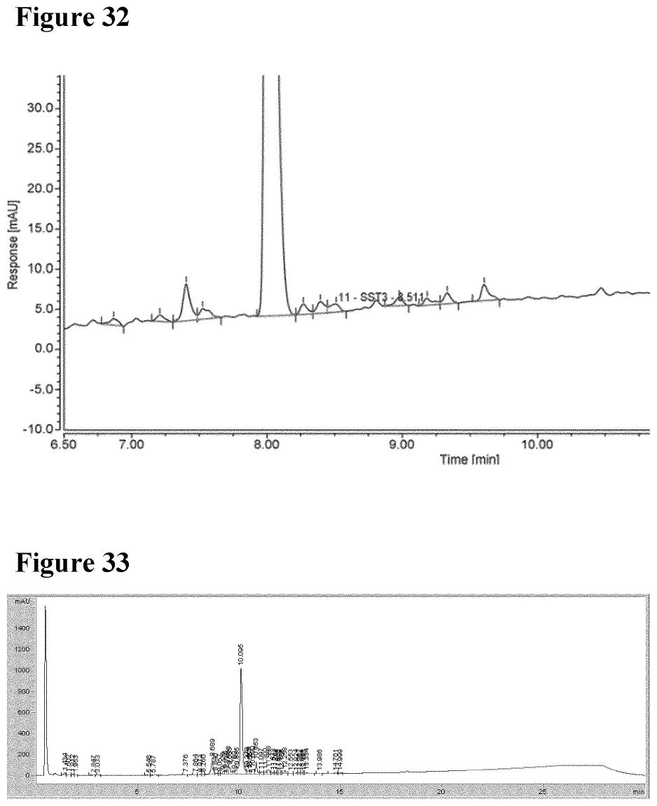

FIGS. 31 and 32 show high performance liquid chromatograph of SPPS synthesis of H₂N-IIKKSTALL-CONH₂ with DMSO:MeCN: 2-MeTHF (8:60:32) as the coupling solvent and DIC/Oxyma pure as the activator system at 60° C. as described above. The purity of the product peak is 86%. The experiment shows that the solvent system is not limited to room temperature but also works at 60° C.

### Reference Syntheses in DMF

Sequence: NH₂-VYWTSPFMKLIHEQCNRADG-CONH₂

The peptide (sequence NH₂-VYWTSPFMKLIHEQCNRADG-CONH₂) was synthesized by SPPS as described in the general procedure using a Tentagel R-RAM resin and DMF as the main solvent. The amino acid building blocks were dissolved in 0.25M HOAt in DMF, HATU (0.25M in DMF) was used as the activator and DIPEA (1.0M in DMF) was used as the base. Capping was performed by addition of AcOH (0.25M in 0.25M HOAt in DMF), HATU (0.25M in DMF, 2 mL) and DIPEA (1.0M in DMF, 1 mL) to the resin and incubation for 5 min at room temperature. The product was obtained as a colorless solid with a crude purity of 51%.

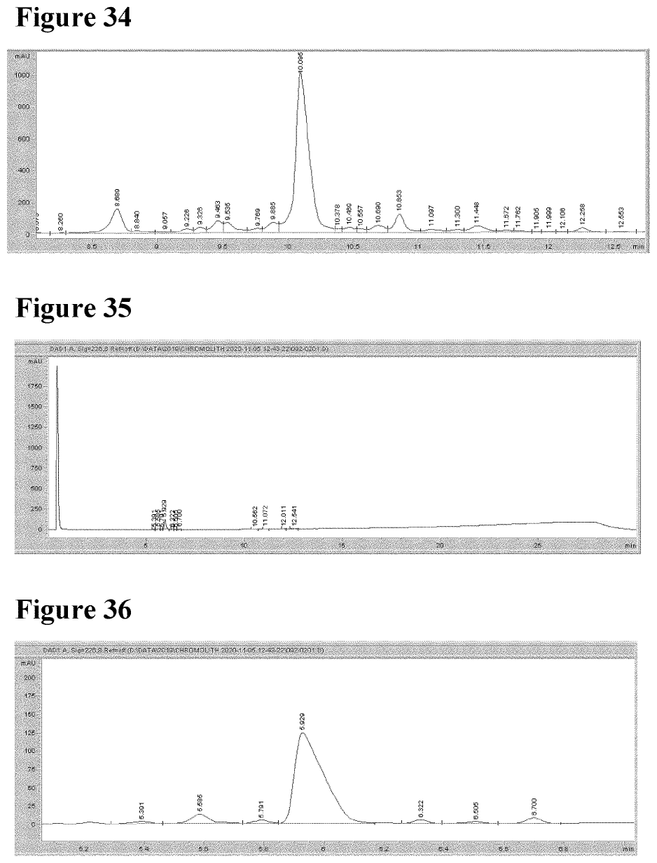

FIGS. 33 and 34 show high performance liquid chromatograph of SPPS synthesis of NH₂-VYWTSPFMKLIHEQCNRADG-CONH₂ with DMF as the solvent and a Tentagel R-RAM resin as described above. The purity of the product peak is 51%. The process uses the current standard solvent DMF and is used a reference for our novel process.

Sequence: NH₂-IIKKSTALL-CONH₂

The peptide (sequence NH₂-IIKKSTALL-CONH₂) was synthesized by SPPS as described in the general procedure using a Tentagel R-RAM resin and DMF as the main solvent. The amino acid building blocks were dissolved in 0.25M HOAt in DMF, HATU (0.25M in DMF) was used as the activator and DIPEA (1.0M in DMF) was used as the base. Capping was performed by addition of AcOH (0.25M in 0.25M HOAt in DMF), HATU (0.25M in DMF, 2 mL) and DIPEA (1.0M in DMF, 1 mL) to the resin and incubation for 5 min at room temperature. The product was obtained as a colorless solid with a crude purity of 78%.

FIGS. 35 and 36 show high performance liquid chromatograph of SPPS synthesis of NH₂-IIKKSTALL-CONH₂ with DMF as the solvent and a Tentagel R-RAM resin as described above. The purity of the product peak is 78%. The process uses the current standard solvent DMF and is used a reference for our novel process. NH₂-IIKKSTALL-CONH₂ and H-IIKKSTALL-NH₂ can be used interchangeable.

CAT Results (determination of the level of epimerization)
CAT Results

| DMF | 2-MeTHF/DMSO/MeCN |
|---|---|
| Crude purity: 51% | Crude purity: 55% |
| Alanine <0.10% D-Enantiomer | Alanine <0.10% D-Enantiomer |
| Valine 0.10% D-Enantiomer | Valine 0.11% D-Enantiomer |
| Threonine <0.10% D-Threonine | Threonine <0.10% D-Threonine |
| Threonine <0.10% D-allo | Threonine <0.10% D-allo |
| Threonine | Threonine |
| Threonine <0.10% L-allo | Threonine <0.10% L-allo |
| Threonine | Threonine |
| Isoleucine <0.10% D-Isoleucine | Isoleucine <0.10% D-Isoleucine |
| Isoleucine <0.10% D-allo | Isoleucine <0.10% D-allo |
| Isoleucine | Isoleucine |
| Isoleucine <0.10% L-allo | Isoleucine <0.10% L-allo |
| Isoleucine | Isoleucine |
| Proline <0.10% D-Enantiomer | Proline <0.10% D-Enantiomer |
| Leucine <0.10% D-Enantiomer | Leucine 0.11% D-Enantiomer |
| Serine <0.10% D-Enantiomer | Serine <0.10% D-Enantiomer |
| Cysteine 0.15% D-Enantiomer | Cysteine 0.19% D-Enantiomer |
| Aspartic acid 0.33% D- | Aspartic acid 2.35% D- |
| Enantiomer | Enantiomer |
| Methionine 0.10% D-Enantiomer | Methionine 0.10% D-Enantiomer |
| Phenylalanine 0.10% D- | Phenylalanine <0.10% D- |
| Enantiomer | Enantiomer |
| Glutamic acid 0.79% D- | Glutamic acid 0.82% D- |
| Enantiomer | Enantiomer |
| Tyrosine 0.15% D-Enantiomer | Tyrosine 0.14% D-Enantiomer |
| Lysine <0.10% D-Enantiomer | Lysine <0.10% D-Enantiomer |
| Arginine <0.10% D-Enantiomer | Arginine 0.13% D-Enantiomer |
| Tryptophan <0.10% D-Enantiomer | Tryptophan <0.10% D-Enantiomer |
| Histidine 0.20% D-Enantiomer | Histidine 0.21% D-Enantiomer |

The table shows the amount of racemization for every amino acid in the peptide NH₂-VYWTSPFMKLIHEQCNRADG-CONH₂ (all natural amino acids). The left column displays the results for a peptide synthesized by current standard protocol (DMF). The right column shows the results for a peptide synthesized by the novel protocol according to the invention (2-MeTHF/DMSO/MeCN). The level of racemization is very similar in both cases and the novel process shows no disadvantage concerning this issue. Furthermore, it circumvents the use of teratogenic solvents.

1. A solid phase peptide synthesis (SPPS) solvent system comprising or consisting of a solvent mixture, wherein the solvent mixture comprises or consists of a first component, a second component and optionally a third component,
wherein the first component is acetonitrile (MeCN),
wherein the second component is dimethylsulfoxide (DMSO) or a tetrahydrofuran based solvent, and wherein the third component is the tetrahydrofuran based solvent in case of the second component is dimethylsulfoxide or wherein the third component is dimethylsulfoxide in case of the second component is the tetrahydrofuran based solvent.

2. The SPPS solvent system of claim 2, wherein said solvent system or solvent mixture is free of a solvent, which is selected from a group consisting of N,N-dimethylformamide (DMF), N,N-dimethylacetamide (DMA), N-methyl-2-pyrrolidon (NMP), dichloromethane (DCM) and mixtures thereof.

3. The SPPS solvent system of claim 1, wherein said solvent system or solvent mixture is free of Diisopropylcarbodiimid (DIC).

4. The SPPS solvent system of claim 1, wherein the solvent mixture comprises or consists of the first component and the second component,
wherein the first component is acetonitrile (MeCN), and
wherein the second component is dimethylsulfoxide (DMSO).

5. The SPPS solvent system of claim 1, wherein the solvent mixture comprises or consists of the first component and the second component,
wherein the first component is acetonitrile (MeCN), and
wherein the second component is 2-methyltetrahydrofuran (2-MeTHF).

6. The SPPS solvent system of claim 1, wherein the solvent mixture comprises or consists of the first component, the second component and the third component,
wherein the first component is acetonitrile (MeCN),
wherein the second component is dimethylsulfoxide (DMSO),
wherein the third component is a tetrahydrofuran based solvent, and
wherein the tetrahydrofuran based solvent is 2-methyltetrahydrofuran (2-MeTHF).

7. The SPPS solvent system of claim 1, wherein the solvent mixture comprises or consists of the first component and the second component, wherein the ratio of the first component and the second component is in the range of from 4:1 to 2:3.

8. The SPPS solvent system of claim 1, wherein the solvent mixture comprises or consists of the first component, the second component and the third component,
wherein the content of the first component is in the range from 40 Vol % to 80 Vol %,
wherein the content of the second component and optional third component are in the range from 20 Vol % to 60 Vol %,
wherein the content of the first, second and optional third components is 100 Vol %.

9. The SPPS solvent system of claim 1, wherein the content of the second component is selected form the group consisting of 20 Vol %, 25%, 30%, 35%, 40%, 45%, 50%, 55% and 60%,
wherein the content of the third component is selected form the group consisting of 20%, 25%, 30%, 35%, 40%, 45%, 50%, 55% and 60%, and
wherein the sum of the contents of the second and third components is 20% to 60%.

10. (canceled)

11. A solid phase peptide synthesis (SPPS) comprising
A) a coupling step by coupling a carboxyl group of one amino acid unit to an amino group of another amino acid unit,
wherein the SPPS comprises the solid phase peptide synthesis (SPPS) solvent system according to claim 1.

12. A kit comprising a solid phase peptide synthesis (SPPS) solvent system according to claim 1.

13. (canceled)

14. A peptide synthesizer comprising the solid phase peptide synthesis (SPPS) solvent system according to claim 1.

15. (canceled)


SEQUENCE LISTING (4 sequences)

SEQ ID NO: 1 - protein, 9 aa, synthetic construct
IIKKSTALL

SEQ ID NO: 2 - protein, 5 aa, synthetic construct
VHLTK

SEQ ID NO: 3 - protein, 40 aa, synthetic construct
KKNKRNTNRRPQDVKFPGGGQIVGGVYLLPRRGPRLGVRA

SEQ ID NO: 4 - protein, 20 aa, synthetic construct
VYWTSPFMKLIHEQCNRADG